(19) United States
(12) Patent Application Publication
(10) Pub. No.: US 2022/0259630 A1
(43) Pub. Date: Aug. 18, 2022

(54) IMPROVED METHOD FOR PRODUCING ISOMALTOOLIGOSACCHARIDES

(71) Applicant: DANISCO US INC, Palo alto, CA (US)

(72) Inventor: BART C KOOPS, PIJNACKER (NL)

(21) Appl. No.: 17/628,094

(22) PCT Filed: Jul. 16, 2020
(86) PCT No.: PCT/US2020/042367

§ 371 Date: Jan. 18, 2022

Related U.S. Application Data
(60) Provisional application No. 63/006,818, filed on Apr. 8, 2020.
(60) Provisional application No. 62/874,541, filed on Jul. 16, 2019.

(51) Int. Cl. C12P 19/12; C12P 19/14; C12N 9/24; C13K 13/00

(52) U.S. Cl. CPC C12P 19/12; C12P 19/14; C12N 9/2402; C13K 13/00; C12Y 302/01002; C12Y 302/0101; C12Y 302/01041; C12Y 302/01001

(57) ABSTRACT

Described is a method for producing improved isomalto-oligosaccharides (IMO) from maltodextrins. The improved method involves the complete or partial replacement of β-amylase, as used in a conventional method, with a selected α-amylase. The resulting IMO have longer chain-length and reduced residual glucose content compared to IMO produced using a conventional method.

### TECHNICAL FIELD

The present method is for producing improved isomalto-oligosaccharides (IMO) from maltodextrins. The improved method involves the complete or partial replacement of β-amylase, as used in a conventional method, with a selected α-amylase. The resulting IMO have longer chain-length and reduced residual glucose content compared to IMO produced using a conventional method.

### BACKGROUND

Isomalto-oligosaccharides (IMO) are partially-digestible sugar-based food ingredients that offer health benefits to humans and other animals. IMO are metabolized to a lower extent than more widely used sugars, such as glucose, fructose and sucrose, thereby providing texture and sweetness benefits at the cost of fewer calories compared to metabolizable sugars. IMO may also supply intestinal bacterial flora with a carbon source affecting the proliferation of desirable bacterial subpopulations. IMO appear to stimulate the production of short-chain fatty acids in the intestine, lowering the intra-luminal pH and inhibiting the growth and activity of enteropathogens. IMO have a low glycemic index, making them desirable for consumption by diabetics, and are not metabolized by most oral bacteria, making the desirable for avoiding cavities.

Chemically, IMO is a mixture of different oligosaccharides, and glucose, that is produced from maltodextrins. The mixture consists of linear oligosaccharide (malto-oligosaccharides) and branched oligosaccharides (isomalto-oligosacharides). IMO is conventionally produced from maltodextrins by the sequential or simultaneous action of a β-amylase and a transglucosidase. The β-amylase produces maltose from the maltodextrins, which is a substrate for the transglucosidase. Maltose is the donor molecule in the transglycolysation reaction, which hydrolyzes maltose, releasing one free glucose molecule and transferring the other glucose molecule to an acceptor.

The acceptor can be another maltose molecule, resulting in a trisaccharide. The most abundant trisaccharide formed is panose. The glucose can also be transferred to a higher sugar, resulting in longer chain isomalto-oligosaccharide, transferred to glucose, resulting in isomaltose formation, or transferred to water, releasing it as another free glucose molecule. The rate at which different oligosaccharides are formed depends on the concentration of the different acceptors. Initially in the reaction, there is a high maltose concentration, resulting primarily in the formation of panose. Later in the reaction, when the maltose concentration is reduced, and the panose concentration increased, the formation of a tetrasaccharide (Glc(α-1,6)Glc(α-1,6)Glc(α-1,4)Glc), will be the more likely reaction product.

Unfortunately, every time a glucose molecule is transferred to an acceptor, a free glucose molecule is released from the donor maltose molecule. This results in IMO syrups with a relatively high glucose content. While glucose can be removed from an IMO syrup via chromatography, the process is costly. The need exists for ways to decrease the amount of glucose present in IMO syrups made from starch substrates and to improve the overall quality of IMO.

### SUMMARY

Described is an improved process for making isomalto-oligosaccharides (IMO) that can result in longer-chain IMO and/or reduced amounts of glucose. Aspects and embodiments of the compositions and methods are described in the following, independently-numbered paragraphs.

1. In a first aspect, an improved method for producing isomalto-oligosaccharides (IMO) from maltodextrins is provided, comprising the steps of: (i) contacting maltodextrins with an α-amylase to produce malto-oligosaccharides, and (ii) contacting the malto-oligosaccharides with a transglucosidase to produce IMO, wherein the method produces longer chain IMO and/or reduced amounts of glucose compared to a method for producing IMO from maltodextrins using β-amylase in step (i).

2. In some embodiments of the method of paragraph 1, step (i) is performed in the presence of no more than 660 diastatic power (DP°) units β-amylase per kg dry weight maltodextrins.

3. In some embodiments of the method of paragraph 1, step (i) is performed in the presence of no more than 264 diastatic power (DP°) units per kg dry weight malto-oligosaccharides.

4. In some embodiments of the method of paragraph 1, step (i) is performed in the presence of no more than 132 diastatic power (DP°) units per kg dry weight malto-oligosaccharides.

5. In some embodiments of the method of paragraph 1, step (i) is performed in the presence of no more than 66 diastatic power (DP°) units per kg dry weight malto-oligosaccharides.

6. In some embodiments of the method of paragraph 1, step (i) is performed in the absence of a β-amylase.

7. In some embodiments of the method of any of paragraphs 1-6, step (i) is performed using an α-amylase that produces malto-oligosaccharides that comprise at least 15% DP3.

8. In some embodiments of the method of any of paragraphs 1-7, step (i) is performed using an α-amylase that produces malto-oligosaccharides that comprise at least 10% DP4.

9. In some embodiments of the method of any of paragraphs 1-8, step (i) is performed using an α-amylase that produces malto-oligosaccharides that comprise at least 5% DP5.

10. In some embodiments of the method of any of paragraphs 1-9, step (i) is performed using an α-amylase that produces malto-oligosaccharides that comprise no more than 40% DP2.

11. In some embodiments of the method of any of paragraphs 1-10, step (i) is performed in the presence of a pullulanase.

12. In some embodiments of the method of any of paragraphs 1-11, steps (i) and (ii) are performed sequentially.

13. In some embodiments of the method of any of paragraphs 1-11, steps (i) and (ii) are performed simultaneously.

14. In some embodiments of the method of any of paragraphs 1-13, the maltodextrins are prepared from a starch-containing substrate using a liquefying α-amylase.

15. In some embodiments of the method of paragraph 14, the liquefying α-amylase and the α-amylase used in step (i) are the same.

16. In another aspect, an improved method for producing isomalto-oligosaccharides (IMO) is provided, comprising the steps of (i) contacting a starch-containing substrate with a liquifying α-amylase to produce maltodextrins, (ii) contacting the maltodextrins with a DP3+ generating α-amylase to produce malto-oligosaccharides and (iii) contacting the malto-oligosaccharides with a transglucosidase to produce IMO having longer chains compared to IMO produced using β-amylase instead of DP3+ generating α-amylase in step (ii).

17. In some embodiments of the method of paragraph 16, the DP3+ generating α-amylase produces malto-oligosaccharides comprising at least 15% DP3, at least 10% DP4, at least 5% DP5, and/or no more than 40% DP2.

18. In some embodiments of the method of paragraph 16 or 17, steps (i) and (ii), and/or steps (ii) and (iii), are sequential, overlapping or simultaneous.

19. In some embodiments of the method of any of paragraphs 16-18, step (ii) is performed in the presence of no more than 660, no more than 264, no more than 132, or no more than 66 diastatic power (DP°) units β-amylase per kg dry weight maltodextrins

20. In some embodiments of the method of any of paragraphs 16-19, step (ii) is performed in the absence of a β-amylase.

21. In some embodiments of the method of any of paragraphs 16-20, step (ii) is performed in the presence of a pullulanase.

22. In some embodiments of the method of any of paragraphs 16-21, the liquefying α-amylase and the DP3+ generating α-amylase used in step (ii) are the same.

23. In another aspect, IMO produced by the method of any of paragraphs 1-22 is provided.

These and other aspects and embodiments of present compositions and methods will be apparent from the description and accompanying drawings.

### BRIEF DESCRIPTION OF THE DRAWINGS

FIG. 1 is a flowchart illustrating the steps and enzymes involved in a conventional process for preparing IMO.

FIG. 2 is a flowchart illustrating the steps and enzymes involved in the present improved process for preparing IMO.

FIG. 3 is a diagram illustrating transglucosidase reactions that occur in a conventional process for preparing IMO.

FIG. 4 is a diagram illustrating reactions between a transglucosidase-glucose complex and malto-oligosacchade acceptor molecules to produce isomalto-oligosacchades.

FIG. 5 is a diagram illustrating reactions between malto-oligosacchade donor molecules and transglucosidase to generate transglucosidase-glucose complexes that can react with malto-oligosacchade acceptor molecules to produce isomalto-oligosacchades.

### DETAILED DESCRIPTION

### I. Definitions

Prior to describing the present process and compositions in detail, the following terms are defined for clarity. Terms not defined should be accorded their ordinary meanings as used in the relevant art.

As used herein the term “starch” refers to any material comprised of the complex polysaccharide carbohydrates of plants, comprised of amylose and/or amylopectin with the formula (C₆H₁₀O₅)ₓ, wherein X can be any number. In particular, the term refers to any plant-based material including but not limited to grains, grasses, tubers and roots and more specifically wheat, barley, corn, rye, rice, sorghum, legumes, cassava, millet, potato, sweet potato, and tapioca. After purification of the complex polysaccharide carbohydrates from the other plant components, it is called “refined starch.”

The term “granular starch” refers to uncooked (raw) starch, which has not been subject to gelatinization.

As used herein, “maltodextrins” refer to oligosaccharides that are generally produced from starch by partial chemical or enzymatic hydrolysis. The size of the polysaccharides generally ranges from DP3 to DP20, but can be longer.

As used herein, “malto-oligosaccharides” refers to oligosaccharides of glucose linked via α-D-1,4 bonds. Exemplary malto-oligosaccharides, and their condensed IUPAC name (refer to IUPAC terminology recommended by the IUB-IUPAC Joint Committee on Biochemical Nomenclature (JCBN) (1982) J. Biol. Chem. 257:3347-51), include but are not limited to, maltose (Glc(α-1,4)Glc), maltotriose (Glc(α-1,4)Glc(α-1,4)Glc), and maltotetraose (Glc(α-1,4)Glc(α-1,4)Glc(α-1,4)Glc).

As used herein, “isomalto-oligosaccharides (IMO)” generally refer to oligosaccharides of glucose that include α-D-1,6 bonds. Exemplary isomalto-oligosaccharides, and their condensed IUPAC name (Id.), include but are not limited to, isomaltose (Glc(α-1,6)Glc), isomaltotriose (Glc(α-1,6)Glc(α-1,6)Glc), and isomaltotetraose (Glc(α-1,6)Glc(α-1,6)Glc(α-1,6)Glc). Branched oligosaccharides having both α-D-1,4 and α-D-1,6 bonds, for example panose (Glc(α-1,6)Glc(α-1,4)Glc) are often considered IMO as well. As used herein, IMO may include some α-D-1,4 bonds.

As used herein, the phrase “degree of polymerization” (DP) refers to the number (n) of anhydroglucopyranose units in a given saccharide. An examples of DP1 is the monosaccharides glucose. Examples of DP2 are the disaccharides maltose and isomaltose.

As used herein, an “α-amylase” is an endo-acting enzyme having the systematic name α-D-(1→4)-glucan glucanohydrolase) and the Enzyme Commission designation EC 3.2.1.1.

As used herein, a “starch processing enzyme” is an enzyme that depolymerizes a starch substrate (including maltodextrin). Exemplary starch processing enzymes are α-amylase, glucoamylase, β-amylase, pullulanase, and α-glucosidase.

As used herein, a “maltogenetic enzyme” is an enzyme that produces mainly maltose as products. Such enzymes include exo-acting enzymes of the classifications EC 3.2.1.2, Some predominantly endo-acting enzymes such as maltogenic α-amylases (EC 3.2.1.133) also produce significant amounts of maltose and will be considered to be “maltogenetic enzymes” for the present purposes.

As used herein, a “maltooligosaccharide producing enzyme” is an enzyme that produced mainly malto-oligsaccharides with a degree of polymerization of longer than 2. Such enzymes include but are not limited to EC 3.2.1.1, EC 3.2.1.116, EC 3.2.1.60 and 3.2.1.98.

As used herein, a “transglucosidase” is synonymous with the term α-glucosidase and the systematic name α-D-glucoside glucohydrolase, having the Enzyme Commission designation EC 3.2.1.20.

As used herein, a “pullulanase” is synonymous with the systematic name α-dextrin endo-1,6-alpha-glucosidase, having the CAZY enzyme database designation EC 3.2.1.41. Other debranching enzymes such as isoamylases (EC 3.2.1.68), having activity on branched maltodextrins, are considered “pullalanases” for the present purposes.

As used herein, “contacting” an enzyme with a substrate refers to bringing the enzyme and substrate together in a common aqueous environment, typically accompanied by mixing to achieve uniform distribution. The term “contacted” is used interchangeably with “treated.”

As used herein, “generating” refers to producing a reaction product as the result of an enzymatic process.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:
° C. degrees Centigrade
BBA barley β-amylase
DE dextrose equivalents
DP degree of polymerization
DP° degrees diastatic power (units of β-amylase activity)
DP3+ DP3 or longer
DPn DP with unknown value
DS dry solids
g or gm gram
HPAE high-performance anion-exchange chromatography
HIPLC high performance liquid chromatography
hr hour
IM2 isomaltose
IM3 isomaltotriose
IM4 isomaltotetraose
IM5 isomaltopentaose
IM6 isomaltohexaose
IM7 isomaltoheptaose
IMO isomalto-oligosaccharides
IUPAC International Union of Pure and Applied Chemistry
kg kilogram
M molar
mg milligram
min minute
mL and ml milliliter
mm millimeter
mM millimolar
MT metric ton
NaAc sodium acetate
NaOH sodium hydroxide
PAD pulsed amperometric detection
PU pullulanase
RI refractive index
RPM or rpm revolutions-per minute
TG transglucosidase
U or u unit
w/v weight/volume
μg microgram
μL and μl microliter
μm micrometer
NM micromolar

### II. Enzymatic Process for Making Improved IMO

Described is an improved enzymatic process for producing isomalto-oligosaccharides (IMO) from maltodextrins using transglucosidase that utilizes α-amylase instead of β-amylase. The improved process results in longer-chain IMO with reduced amounts of glucose compared to conventional methods, making possible the more economical production of high-IMO, low-glucose specialty syrups.

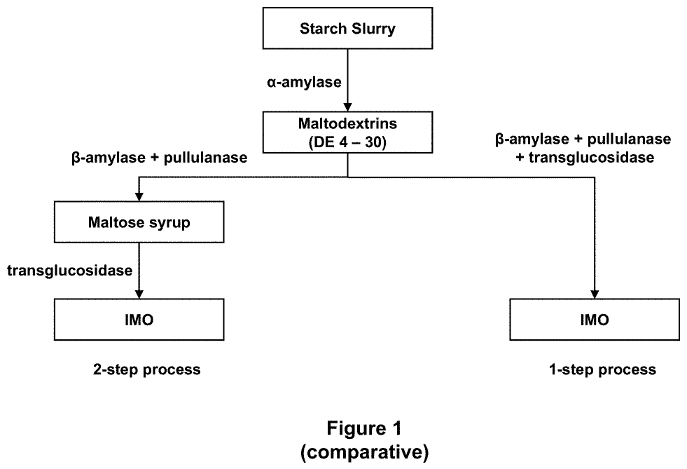

The improved process may be visualized with the aid of the accompany drawings. As shown in the flowchart in FIG. 1, IMO is conventionally produced from maltodextrins by the sequential or simultaneous action of a β-amylase and a transglucosidase. In the conventional two-step process (left side of flowchart), a starch slurry is converted to maltodextrins in a liquefaction process and the maltodextrins are treated with β-amylase and pullulanase to produce a maltose syrup, which is then treated with transglucosidase to produce IMO. In the conventional one-step process (right side of flowchart), a starch slurry is converted to maltodextrins in a liquefaction process and the maltodextrins are treated simultaneously with β-amylase, pullulanase and transglucosidase to produce IMO, without isolating or separating the maltose syrup.

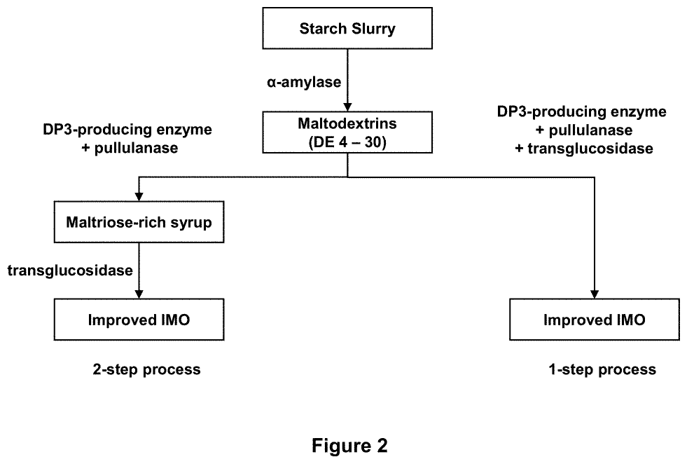

As shown in the flowchart in FIG. 2, the improved process may continue to begin with the conversion of starch slurry to maltodextrins in a liquefaction process. However, matodextrins are now treated with an enzyme capable of generating malto-oligosaccharides longer than DP2 (i.e., maltose). Instead, a DP3 (or longer malto-oligosaccharide)-producing enzyme and pullulanase are used to produce a maltotriose (or longer oligosaccharide)-rich syrup, which is then treated with transglucosidase to produce improved IMO in a two-step process (left side of flowchart). Alternatively, the maltodextrins are treated simultaneously with a DP3 (or longer malto-oligosaccharide)-producing enzyme, pullulanase and transglucosidase to produce improved IMO, without isolating or separating maltotriose (or longer malto-oligosaccharide)-rich syrup in a one-step process (right side of flowchart).

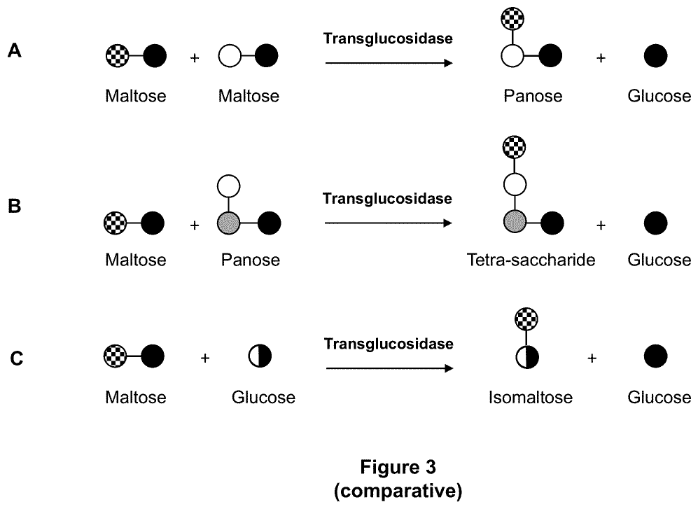

The advantages of the improved method will be apparent upon examining end-products of the transglucosidase reactions. In FIGS. 3-5, glucose molecules are represented by circles and glycosidic bonds are represented by lines connecting the circles. Glucose molecules having reducing ends are filled solid black, donor glucose molecules are filled with a checked pattern, glucose acceptor glucose molecules are filled solid white, and non-reacting glucose molecules, are shown in grey. Free glucose molecules having a reducing end, but which also serve as acceptor glucose molecules, are filled half-white and half-black.

In the conventional process, particularly early in the stages of the transglycosilation reaction, maltose produced from the starch hydrolysate by β-amylase is abundant and serves as both the predominant donor molecule and acceptor molecule for the transglucosidase (FIG. 3A). This results in the production of a trisaccharide, the most abundant being panose, along with a free glucose. Later in the reaction, when maltose becomes depleted, longer acceptor molecules are relatively more abundant, producing longer IMO, along with a free glucose (FIG. 3B). Free glucose itself can also serve as an acceptor for transglucosidase (FIG. 3C), in which case a short IMO is product, but again with the release of what is merely a different free glucose.

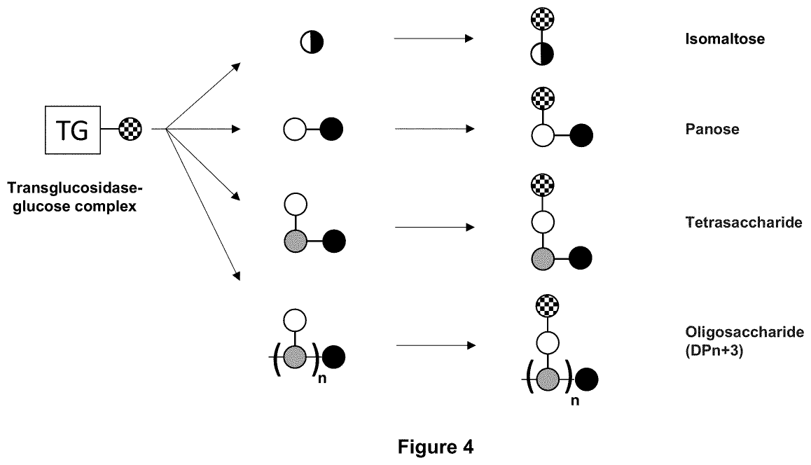

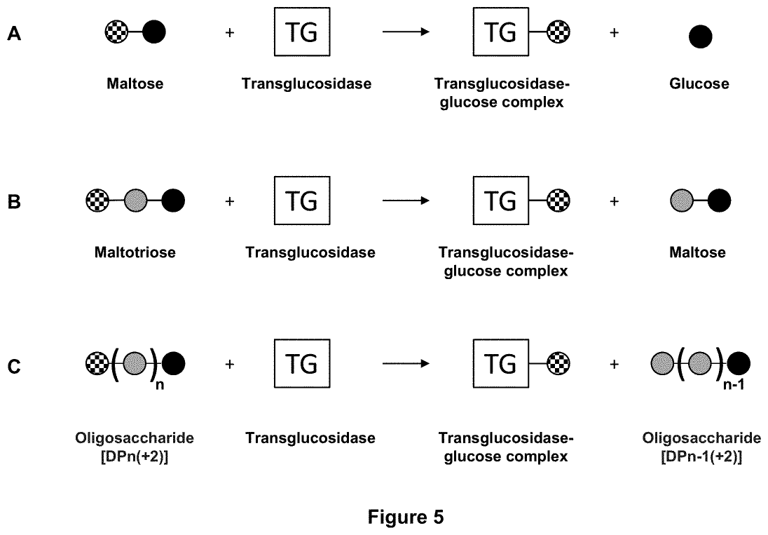

FIG. 5 illustrates the advantages of the improved process. As before, when maltose is the donor malto-oligosaccharide as in a convention IMO production process, a free glucose is formed with every transglucosidase reaction (FIG. 5A). In contrast, when, a longer donor malto-oligosaccharide is used, such as maltotriose (FIG. 5B), maltotetraose (FIG. 5C), or higher, the transglucosidase reaction produces no free glucose during the first part of the reaction which forms the transglucosidase-glucose complex. Transglucosidase-glucose complex formed as in FIGS. 4B and 4C can interact with acceptor oligosaccharides of various lengths to produce IMO. No free glucose is generated in this part of the reaction (FIG. 5).

### III. Enzymatic Compositions

A. DP3+ Generating α-Amylases

DP3+ generating (also called DP3+ producing) α-amylases suitable for producing malto-oligosaccharides for use in the improved process are those that produce malto-oligosaccharides longer than DP2 (i.e., maltose) from maltodextrins. Such enzymes produce DP3, DP4, DP5, or longer, malto-oligosaccharides. Enzymes that produce significant amounts of DP3 include, but are not limited to, α-amylases from Aspergillus, e.g., A. kawachi, A clavatus and A. oryzae. Maltotriose-producing amylases have been identified in Streptomyces griseus, Bacillus subtilis, Microbacterium imperiaie and Chloroflexus aurantiacus. Enzymes that produce significant amounts of DP4 include, but are not limited to, an amylase from Pseudomonas saccharophila. Enzymes that produce significant amounts of DP5 include, but are not limited to, α-amylases from several Bacillus spp., including B. stearothermophilus and B. licheniformis, as well as enzymes from Cytophaga spp.

Generally, a DP3+ generating α-amylase suitable for use according to the present methods is any α-amylase that produces a sugar profile that (when the reaction is left to process for sufficient time) has a minimum of 15% DP3, a minimum of 10% DP4 or a minimum of 5% DP5, along with a maximum of 40%, a maximum of 30%, a maximum of 20%, a maximum of 10% or even a maximum of 5% DP2. More than one DP3+ generating α-amylase can be used, in which case the combination of DP3+ generating α-amylases produces the described profile of malto-oligosaccharides.

B. Transglucosidases

The second enzyme critical to the method for producing improved isomalto-oligosaccharides (IMO) from malto-oligosaccharides is transglucosidase, also known as α-glucosidase and α-D-glucoside glucohydrolase. The molecules are classified as EC 3.2.1.20 enzymes in CAZy Family GH31 and have been identified in numerous organisms. Genbank includes over 400 entries for transglucosidases.

The enzyme exemplified herein is from Aspergillis niger and is expressed in Trichoderma reesei. The enzyme expresses at high levels but is otherwise not recognized as having unique properties compared to other transglucosidases studied. Accordingly, a large number of transglucosidases, derived from many organisms, are believed to be suitable for producing isomalto-oligosaccharides (IMO) from maltodextrins.

The exemplified enzyme is commercially-available as TRANSGLUCOSIDASE L2000® (DuPont Nutrition & Biosciences) with an activity of 1700 transglucosidase units (TGU)/g. One TGU is defined as the amount of enzyme required to produce one micromole of panose per minute under the conditions of the assay. A minimum of 0.1 kg/MT of TRANSGLUCOSIDASE L2000®/MT of DS is needed. In all the work described herein, 1 kg/MT DS was used.

C. Liquefying α-Amylase

Liquefying α-amylase for converting crude feedstocks, such as a starch from grains and other plant materials, to maltodextrins are well known in the art and include enzymes derived from numerous microorganisms. Exemplary enzymes are commercially available as, e.g., FUELZYME™ (BASF Enzymes LLC, San Diego, Calif.), LPHERA®, AVANTEC® and LIQUOZYME® products (Novozymes) and; SPEZYME® products (DuPont). More than one liquefying α-amylase can be used.

In some embodiments, the liquefying α-amylase may additionally be useful as the DP3+ generating enzyme for use in the improved process, depending on the profile of malto-oligomers generated. Accordingly, the liquefying α-amylase(s) may be, or may include, the DP3+ generating enzyme(s).

The enzyme concentration needed to produce such a sugar profile depends on the type of reaction products it produces, the reaction conditions and the reaction time. A trained person can determine the optimal amount. As an example, SPEZYME® ALPHA PF dosed of 0.2 kg/MT DS on a 12 DE liquefact can produce a syrup with over 20% DP5 in about 7 hours.

Starch liquefaction can be performed above, at or below the gelatinization temperature of the starch substrate. Other enzymes may be present, e.g., proteases.

D. Enzyme Blends for Performing the Improved Process

Suitable enzyme blends are those that produce, from a 12 DE starch liquefact in the absence of transglucosidase, a syrup with a high content of DP3-DP5 malto-oligosaccharides. In this context, a high content means that a minimum of 15%, 25%, 30%, 35%, 40%, 45%, 50%, 55%, 60% of the total malto-oligosaccharides are DP3, DP4 and/or DP5. Another way of defining a high content syrup is by individual sugar components, where DP3 is minimum 15%, DP4 is minimum 10%, DP5 is minimum 5% and/or DP2 is maximum 40%, 30%, 20%, 10% or even 5%.

Suitable enzyme blends are additionally or alternatively those that produce, from a 12 DE starch liquefact in the presence of transglucosidase, a syrup with content of more than 4% isomaltopentaose, more than 2% isomaltohexaose and/or more than 1% isomaltoheptaose as a percentage of total sugars, as measured as described in the Examples.

E. No Requirement for β-Amylase Activity

A key feature of the improved process and enzymatic compositions is that they are performed substantially in the absence of maltogenic activity with the intent of minimizing the use of maltose as a donor for transglucosidase, thereby reducing the production of free glucose. Substantially in the absence of β-amylase activity means that enzymes categorized as β-amylases or maltogenic amylases, and/or enzyme compositions (such as blends) having β-amylase activity, are not necessary or required to produce the improved IMO described herein. Accordingly, no β-amylase and/or beta-amylase activity need be applied to maltodextrins for the purpose of producing improved IMO as described.

β-amylase activity is typically expressed in degrees diastatic power (DP°). One unit of diastase activity, expressed as degrees DP (DP°), is defined as the amount of enzyme, contained in 0.1 ml of a 5% solution of the sample enzyme preparation, that will produce sufficient reducing sugars to reduce 5 mL of Fehling's solution when the sample is incubated with 100 mL of the substrate for 1 hour at 20° C. The reducing sugar groups produced during the reaction are measured in a titrimetric procedure using alkaline ferricyanide. This enzyme assay measures the activity of both α- and β-amylases present in a given sample.

The amount of β-amylase activity that can be tolerated in the present improved methods, such that the described advantages in IMO quality are still realized, is maximally about 660 DP° units β-amylase per kg DS starch hydrolysate, maximally about 264 DP° units β-amylase per kg DS starch hydrolysate, maximally about 132 DP° units β-amylase per kg DS starch hydrolysate, and maximally about 66 DP° units β-amylase per kg DS starch hydrolysate. As stated, no measurable amount of β-amylase need be present.

F. Raw Starch Hydrolyzing Enzymes

Many starch degrading enzymes are active on raw starch, as described in, e.g., U.S. Pat. Nos. 7,037,704, 7,205,138, 7,303,899, 7,378,256, and reference contained within. These enzymes are generally referred to as raw starch hydrolyzing enzymes or granular starch hydrolyzing enzymes (GSHE). GSHE that liberate DP3 or longer sugars are suitable for use as described. GSHE can be used in a two-step reaction where raw starch is treated with a GSHE, with or without pullulanase, to produce a malto-oligosaccharide which is then reacted with transglucosidase. GSHE can also be used in a one-step reaction where the raw starch is treated with a GSHE, with or without pulluanase, and simultaneously reacted with transglucosidase. Examples of enzymes that can liberate oligosaccharides from raw starch include, but are not limited to, SPEZYME® ALPHA PF, SPEZYME® XTRA, Aspergillus karwachi alpha-amylase, and OPTIMALT® 4G.

### IV. Features and Uses of Improved IMO

The improved process and enzymatic compositions allow the production of isomalto-oligosaccharides (IMO) from maltodextrins for any number of uses. The IMO are longer, and the content of glucose in the syrup is lower, than with a conventional process. The syrup may be physically separated into fractions having a desired DP range, using methods similar to those used for conventional syrup. More specifically, the IMO produced using the present compositions and methods are longer than IMO produced using conventional methods, and the ratios of the content (% of total sugars) of longer IMO molecules over shorter molecules is increased. Longer IMO are likely to be more poorly metabolized, offering greater health benefits to consumers and more food ingredient options to food producers

These and other aspects and embodiments of the present methods, and compositions resulting, therefrom, will be apparent to the skilled person in view of the present description. The following examples are intended to further illustrate, but not limit, the compositions and methods.

### EXAMPLES

### Example 1. Transglycosylation Reactions Using Maltose and Maltotriose

Transglycosylation reactions were performed using reagent grade maltose and reagent grade maltotriose (both purchased from Sigma Aldrich) at 30% DS in water. Solutions of maltose or maltotriose at 30% DS were made and adjusted to pH 4.2. Approximately 2 g of the maltose or maltotriose solution was weighed into Eppendorf tubes. To each Eppendorf tube transglucosidase (TRANSGLUCOSIDASE® L-2000; DuPont) was added at a dose of 1 kg product per MT of substrate DS. The tubes were incubated in a thermostatic mixer (thermoblock) for 48 hr at 60° C. with a shaking speed of 750 rpm.

At appropriate times samples were taken for HPLC analysis. A 100 μl portion was taken from the reaction medium, diluted 10 times with distilled water and boiled. Following filtration, 20 μl was injected into an HPLC apparatus equipped with a Bio-Rad Aminex HPx-42A column (#1250096, 300 mm×7.8 mm). The mobile phase was HPLC grade distilled water, running at 0.6 ml/min for 22.5 min. The temperature of the column was 85° C. and detection was performed using a RI detector with cell temperature of 40° C.

As summarized in Table 1, the majority of maltose and maltotriose had been consumed after 48 hr. The results further show that approximately half-as-much glucose (DP1) was released after 48 hr when the reaction is performed on maltotriose compared to maltose. When maltose is treated with transglucosidase, branched maltotriose (DP3) is initially formed, followed by formation of branched oligosaccharides (DPn) with higher degree of polymerisation, which are likely formed from DP3. When maltotriose is treated with transglucosidase, there is a more rapid formation of higher branched oligosaccharides (DPn), which initially is likely to be DP4. The results in Table 1 show that branched IMO with higher degree of polymerisation are formed by transglucosidase when the reaction starts with maltooligosaccharides of higher degree of polymerization. This also results in decreased glucose formation.

TABLE 1
Production of sugars (%) from maltose
and maltotriose

| Enzymes | Time (hr) | DPn | DP3 | DP2 | DP1 |
|---|---|---|---|---|---|
| Maltose + TG | 0 | 0.0 | 0.0 | 93.9 | 6.1 |
| | 2 | 0.6 | 17.9 | 69.4 | 12.1 |
| | 4 | 2.7 | 30.0 | 49.7 | 17.6 |
| | 6 | 5.3 | 36.4 | 36.8 | 21.5 |
| | 18 | 14.9 | 29.0 | 25.7 | 30.4 |
| | 20 | 15.1 | 27.5 | 26.4 | 31.0 |
| | 24 | 15.4 | 24.8 | 27.7 | 32.2 |
| | 48 | 14.2 | 18.5 | 30.0 | 37.3 |
| Maltotriose + TG | 0 | 9.9 | 84.2 | 5.9 | 0.0 |
| | 2 | 28.6 | 53.8 | 14.5 | 3.0 |
| | 4 | 38.8 | 39.3 | 16.7 | 5.2 |
| | 6 | 43.9 | 32.6 | 16.2 | 7.3 |
| | 18 | 44.3 | 24.1 | 16.6 | 15.0 |
| | 20 | 43.7 | 23.6 | 17.2 | 15.5 |
| | 24 | 42.4 | 22.8 | 18.2 | 16.6 |
| | 48 | 36.5 | 20.3 | 22.2 | 21.0 |

### Example 2. Transglycosylation Reactions Using DP2 and DP4 Syrups

Since it is not commercially attractive to perform the transglycosylation reaction with pure malto-oligosaccharides, the experiment in Example 1 was repeated using a starch hydrolysate rich in DP4. This starch hydrolysate was prepared using a DP4-producing enzyme and was compared to a starch hydrolysate rich in maltose prepared using β-amylase. Both starch hydrolysates were prepared from a corn liquefact with a DE of 11.28 at 32.5% DS. To produce the DP4 rich hydrolysate, the liquefact was incubated with a DP4-producing α-amylase (OPTIMALT® 4G; DuPont) at 0.9 kg/MT DS plus pullulanase (OPTIMAX® L-1000; DuPont) at 0.4 kg/MT DS at pH 5.0 and 60° C. for 48 hr. To produce the maltose rich hydrolysate, the same liquefact was treated with a β-amylase (OPTIMALT® BBA; DuPont) at 0.9 kg/MT DS plus pullulanase at 0.4 kg/MT DS.

At appropriate time points, samples were taken for HPLC analysis. A 100 μl portion was taken from the reaction medium, diluted 10 times with distilled water and boiled. Following filtration, 20 μl was injected into an HPLC apparatus equipped with a Bio-Rad Aminex HPx-42A column (#1250096, 300 mm×7.8 mm). The mobile phase was HPLC-grade distilled water, running at 0.6 ml/min for 22.5 minutes. The temperature of the column was 85° C. and detection was done in a RI detector with cell temperature of 40° C. The sugar compositions of the resulting syrups, following 48 hr of reaction time, are summarized in Table 2.

TABLE 2
Sugar composition of maltose and maltotetraose
syrups produced from corn liquefact

| Sugar | DP2 syrup | DP4 syrup |
|---|---|---|
| DP1 | 2.03 | 5.1 |
| DP2 | 60.2 | 6.1 |
| DP3 | 12.6 | 15.0 |
| DP4 | 2.6 | 43.9 |
| DP5+ | 22.6 | 29.9 |

These syrups were further reacted with transglucosidase to produce IMO. The above syrups rich in DP2 or DP4 were adjusted to pH 4.2 and 30% DS. A 2 g sample of the DP4-rich or DP2-rich syrup was treated with transglucosidase at 1 kg/MT DS for 24 hr at 60° C. After 24 hr, a sample was taken for HPLC analysis. As above, a 100 μl portion was taken from the reaction medium, diluted 10 times with distilled water, boiled subjected to centrifugation and filtered, followed by the injection of 20 μl into an HPLC apparatus equipped with the same Bio-Rad Aminex HPx-42A column using the same mobile phase, flow rate and temperature. The sugar profiles of the starting DP2 and DP4 syrups as well as the sugar profiles obtained from the transglucosidase reaction after 24 hr are shown in Table 3.

TABLE 3
Sugar composition of DP2 and DP4 syrups
before and after TG treatment

| | | DP2 syrup | | DP4 syrup |
|---|---|---|---|---|
| | DP2 | after | DP4 | after |
| Sugar | syrup | TG reaction | syrup | TG reaction |
| DPn | 18.3 | 16.2 | 23.2 | 22.2 |
| DP10 | 0.4 | 0.4 | 0.3 | 0.5 |
| DP9 | 0.4 | 0.4 | 0.3 | 1.0 |
| DP8 | 0.5 | 0.5 | 2.9 | 1.4 |
| DP7 | 0.6 | 0.7 | 1.4 | 2.4 |
| DP6 | 0.9 | 1.6 | 0.9 | 5.0 |
| DP5 | 1.5 | 3.1 | 0.8 | 8.3 |
| DP4 | 2.6 | 10.7 | 43.9 | 10.4 |
| DP3 | 12.6 | 15.5 | 15.0 | 14.5 |
| DP2 | 60.2 | 23.1 | 6.1 | 17.0 |
| DP1 | 2.0 | 27.8 | 5.1 | 17.4 |

The IMO syrup prepared using the DP4-rich hydrolysate is clearly different in composition compared to the syrup prepared using the DP2-rich hydrolysate. DP1 levels are much lower following the TG reaction using the DP4-rich syrup (17%) compared to the DP2-rich syrup (28%). DP2 is also lower following the TG reaction using the DP4-rich syrup, and the longer oligosaccharides, including DP5, DP6, DP7, D8 and DP9, are more abundant.

### Example 3. Selection of Enzymes for the Production of DP2-DP5 Syrups

The experiment described in Example 2 was repeated by applying a more diverse panel of enzymes to maltodextrins to make DP2, DP3, DP4 and DP5-rich syrups from maltodextrins.

The enzyme used for the production of a DP2 rich syrup was either β-amylase (OPTIMALT® BBA, as above) at 0.9 kg/MT DS, plus pullulanse (OPTIMAX® L1000, as above) at 0.4 kg/MT DS, or a maltogenic amylase (OPTIMALT® 2G) at 0.5 kg/MT DS, with or without pullulanase at 0.4 kg/MT DS. The enzymes use for the production of DP3-rich syrup was DP3-producing α-amylase from Aspergillus kawachi (GC626; DuPont) at 0.5 kg/MT DS, with or without pullulanase at 0.4 kg/MT DS. The enzymes use for the production of DP4-rich syrups was the DP4-producing α-amylase (OPTIMALT® 4G, as above) at 0.9 kg/MT DS, with and without pullulanase at 0.4 kg/MT DS. The enzymes used for DP5-rich syrups were either a Cytophaga sp.-based α-amylase at 6 μg purified protein/g DS, or a Bacillus stearothermophilus-based α-amylase (SPEZYME® ALPHA PF) at 0.2 kg/MT DS, with or without pullulanase at 0.4 kg/MT DS.

The aforementioned amylase enzymes will be referred to as BBA, 2G, 626, 4G, CspAmy and PF, respectively, in this example and all further examples. The pullulanse used in all further examples is OPTIMAX® L1000 and the transglucosidase is TRANSGLUCOSIDASE L-2000®, unless otherwise mentioned.

For all reactions, 10 g of corn liquefact was incubated for 48 hr at 60° C. with the above-identified enzymes at the indicated dose and pH. As above, a 100 μl portion was taken from the reaction medium and used to perform HPLC analysis.

In all cases is was apparent that the addition of pullulanase in the reaction was desirable to increase the levels of desired saccharide and reduce the amounts of higher sugars (DPn). Consequently, only results with pullulanase will be discussed. With DP3-producing α-amylase (GC626), a syrup with approximately 32.5% DP3 and 39% DP2 was obtained. With DP4-producing α-amylase (OPTIMALT® 4G), a syrup with approximately 44% DP4 was obtained.

With the DP5-producing α-amylases (CspAmy or SPEZYME® ALPHA PF), syrups with approx. 29% and 21% DP5, respectively, were obtained. The sugar composition (% of total) of all these reactions is shown in Table 4. The enzyme abbreviations used in the Table are readily apparent from the foregoing description.

TABLE 4
Sugar compositions of syrups prior to TG treatment

Enzyme

| Sugar | BBA | 2G | 626 | 4G | CspAmy | PF |
|---|---|---|---|---|---|---|
| DP1 | 2.0 | 7.2 | 11.7 | 5.1 | 8.0 | 3.9 |
| DP2 | 60.2 | 58.2 | 38.9 | 6.1 | 11.1 | 12.8 |
| DP3 | 12.6 | 2.6 | 32.5 | 15.0 | 18.9 | 15.4 |
| DP4 | 2.6 | 4.5 | 4.6 | 43.9 | 8.1 | 8.1 |
| DP5 | 1.5 | 0.8 | 3.6 | 0.8 | 29.1 | 21.0 |
| DP6 | 0.9 | 0.7 | 2.9 | 0.9 | 5.9 | 17.0 |
| DP7 | 0.6 | 0.5 | 0.9 | 1.4 | 2.9 | 2.2 |
| DP8 | 0.5 | 0.6 | 0.4 | 2.9 | 2.8 | 2.6 |
| DP9 | 0.4 | 0.6 | 0.2 | 0.3 | 2.1 | 2.2 |
| DP10 | 0.4 | 0.6 | 0.1 | 0.3 | 2.0 | 1.5 |
| DPn | 18.3 | 23.8 | 4.3 | 23.2 | 9.0 | 13.4 |

In a second step, the above syrups were adjusted to pH 4.2 and 30% DS and 2 g of each was further reacted with transglucosidase at 1 kg/MT DS for 24 hours at 60° C. As above, a 100 μl portion was taken from the reaction medium and used to perform HPLC analysis. A second sample was prepared for analysis by high-performance anion-exchange chromatography using pulsed amperometric detection (HPAE-PAD). Where other methods separate saccharides based on size (monomers, dimers etc.), HPAE-PAD is capable of separating isomers such as maltotriose, panose and isomaltotriose. Specifically, 100 μl sample was taken, diluted 1,000 times and boiled for 10 min. Following filtration, a 10 μl sample was injected on a Carbopac PA200 column (3 mm×250 mm) installed with a guard column at a flow rate of 0.5 ml/min and a temperature 30° C. PAD was performed with a cell temperature of 25° C. During the 60 min chromatographic run the following conditions were used: (i) prior to sample injection, the column was equilibrated for 10 minutes with 10% 1 M NaOH and 10% 500 mM NaOAc and 80% MilliQ water. The separation of the sugars was accomplished by elution with constant 10% 1M sodium hydroxide and 90% MilliQ water for 5 minutes. During the following 5 minutes a gradient with 500 mM NaOAc was started where the % NaOAc in the mobile phase increased from 0 to 8% and % of MilliQ water decreased from 90 to 82%. In the following 50 minutes the gradient changed and % MilliQ in the mobile phase decreased from 82% to 0% and % NaOAc increased from 8 to 90%. The gradient is shown in table 5.

TABLE 5
Chromatographic gradient

| Time | Water | 1M | 500 mM |
|---|---|---|---|
| (min) | (%) | NaOH (%) | NaAc (%) |
| 0-5 | 90 | 10 | |
| 5-10 | 90-82 | 10 | 0-8 |
| 10-60 | 82-0 | 10 | 8-90 |

To calculate IMO content, two analyses were required. With the conventional HPLC method (described, above), the content (%) of the different saccharides is calculate by measuring the area of the DP1, DP2, DP3, DP4, DP5, DP6, DP7, DP8, DP9, DP10 and DPn peaks from the chromatogram. For clarity, 10% DP2 means that 10% by weight of the sugars present in the final sugar composition is DP2 (and so forth). For the purposes of Table 5, DPn refers to ≥DP11. As mentioned, this analysis does not provide information regarding the isomers present. However, the isomers present in, for example, the DP2 peak, can be distinguished by HPAE-PAD analysis. Chromatograms from this HPAE-PAD analysis revealed peaks from different isomers that could be identified based on the separate analysis of a standard sample with known components. Since the concentration of each component in the standard mix is known, the content of that particular component in the sample can be calculated. For example, if a sample contains 1.4% (w/v) maltose, 7.4% (w/v) isomaltose, 2.4% (w/v) kojibiose and 1.6% (w/v) nigerose (w/v) based on HPAE-PAD analysis, this means that the total DP2 contains 11%, maltose, 58% isomaltose, 19% kojibiose and 12% nigerose. If for example the DP2 content in the syrup is 10% (measured by conventional HPLC), this means that the content of the isomers in the total syrup are: 1.1% maltose, 5.8% isomaltose, 1.9% kojibiose and 1.2% nigerose. In this manner, DP1, DP2 and DP3 isomers can be distinguished.

In the case of DP1, DP2 and DP3 sugars, the majority of isomers that are likely to be formed can be affirmatively distinguished since pure components can be purchased from chemical supply companies and used as standards. For the longer oligosaccharides, e.g., DP4 and higher, not all isomers can be distinguished using readily-available standards. Accordingly, in the case of DP4 and higher isomers, only linear malto-oligosaccharides up to DP10 are distinguished, i.e., malto-tetraose, malto-pentaose, malto-hexaose, malto-heptaose, malto-octaose, malto-nonaose and malto-decaose. In addition, linear isomalto-oligosaccharides up to DP7 are distinguished, i.e., isomalto-tetraose, isomalto-pentaose, isomalto-hexaose and isomalto-hetpaose are identified. Other, more complex, branched oligosaccharides show up in the chromatogram as unidentified peaks. As the oligomers become longer, there is also a greater likelihood that the peaks overlap on the chromatogram, making quantitation problematic. The absence of commercially-available standards and methods for separating the longer isomers is understandable as the practical production of these IMO is made possible only in view of the present improved method.

To identify the percentage of the DP4 being isomalto-tetraose, the assumption is made that it only contains malto-tetraose and isomalto-tetraose. Other unidentified branched tetramers are not taken into account for the IMO content calculation. Since branched oligosaccharides are often considered IMO this calculation results in a small underestimation of the total IMO content. This is the same for the longer malto-oligodaccharides. For isomers of DP8-DP11, it is assumed that they all are linear malto-oligosaccharides. This leads again to a small underestimation of the total IMO content.

The results summarized in Table 6 show the sugar composition of the transglucosidase-treated syrups as % of total sugars measured by HPLC and referring to DP number. The results summarized in Table 7 show IMO content in transglucosidase-treated syrups as % of total sugars as measured by HPAE-PAD. In this table IM2 stands for isomaltose, IM3 for isomaltotriose etc.

TABLE 6
Sugar compositions of DP2-DP5 syrups
following transglucosidase treatment

Enzyme

| Sugar | BBA | 2G | 626 | 4G | CspAmy | PF |
|---|---|---|---|---|---|---|
| DP1 | 27.8 | 31.2 | 28.5 | 17.4 | 18.3 | 15.4 |
| DP2 | 23.1 | 22.7 | 24.7 | 17.0 | 18.4 | 15.5 |
| DP3 | 15.5 | 14.2 | 19.0 | 14.5 | 15.9 | 14.4 |
| DP4 | 10.7 | 4.8 | 11.6 | 10.4 | 10.9 | 10.7 |
| DP5 | 3.1 | 2.6 | 6.3 | 8.3 | 8.8 | 9.1 |
| DP6 | 1.6 | 1.0 | 2.9 | 5.0 | 7.7 | 8.5 |
| DP7 | 0.7 | 0.6 | 1.4 | 2.4 | 5.0 | 6.6 |
| DP8 | 0.5 | 0.5 | 0.6 | 1.4 | 3.0 | 3.6 |
| DP9 | 0.4 | 0.5 | 0.3 | 1.0 | 2.1 | 2.2 |
| DP10 | 0.4 | 0.5 | 0.2 | 0.5 | 1.6 | 1.6 |
| DPn | 16.2 | 21.6 | 4.5 | 22.2 | 8.3 | 12.4 |

TABLE 7
IMO content of above syrups as measured by HPAE-PAD

| | Enzyme | | | | | |
|---|---|---|---|---|---|---|
| IMO | BBA | 2G | 626 | 4G | CspAmy | PF |
| IM2 | 12.5 | 12.7 | 13.7 | 8.9 | 11.4 | 9.7 |
| IM3 | 8.5 | 9.3 | 9.8 | 8.0 | 8.4 | 7.0 |
| Panose | 6.2 | 4.9 | 9.2 | 6.5 | 7.5 | 7.5 |
| IM4 | 9.5 | 4.6 | 10.1 | 6.8 | 6.4 | 5.2 |
| IM5 | 1.3 | 2.2 | 1.2 | 1.2 | 0.7 | 0.4 |
| IM6 | 1.6 | 1.0 | 2.4 | 2.8 | 4.5 | 4.3 |
| IM7 | 0.7 | 0.6 | 0.4 | 1.6 | 0.4 | 0.6 |
| Total IMO | 40.3 | 35.2 | 46.7 | 35.8 | 39.2 | 34.8 |

Comparing the final IMO syrups summarized in Table 4, 6 and 7, it is apparent that the longer the donor molecules in the transglucosidase reaction, the lower the increase in DP1 (free glucose) following treatment. In addition, the total DP1 content is lower as the length of donor molecule increases.

In a two-step reaction involving first the production of a malto-oligosaccharide-rich syrup, followed by transglucosidase treatment, there is little or no decrease in DPn levels. This is likely caused by the fact that pullulanase was added in the first reaction and not during the TG reaction.

Following transglucosidase treatment, there is less DP2 present when starting from a DP4 or DP5-rich syrup and hardly any difference in the amount of DP3 and DP4 present after transglucosidase treatment in any of the malto-oligosaccharide-rich syrups. The amount of DP6-DP10 after transglucosidase treatment is increases with increasing length of the donor molecule. Transglucosidase reactions that used syrups that were rich in longer sugars resulted in reduced amounts of isomaltose and increased amounts of isomaltohexaose and isomaltoheptaose.

The IMO content (i.e., the sum of isomaltose, isomaltotriose, pannose, isomaltotetraose, isomaltopentaose, isomaltohexaose and isomaltoheptaose) is higher when staring from a DP3-rich syrup compared to a DP2-rich syrup. This is not the case for DP4 and DP5-rich syrups, which have a higher content of DPn, possibly due to the lack of a debranching activity during the tranglycosylation reaction. If these non-available, presumably branched maltooligosaccharides are be made available by the addition of pullulanse at the time of TG treatment, the IMO content following transglucosidase treatment using longer donor molecules would probably be higher.

### Example 4. Simultaneous Malto-Oligosaccharide Production and Transglucosidation

In conventional IMO production, it is not uncommon to contact maltodextrins with β-amylase and transglucosidase at the same time to product IMOs, in a one-step reaction. In this example the experiments described in Example 3 were repeated, but instead of first producing the malto-oligosaccharide-rich syrups, malto-oligosaccharide production and transglucosidation were performed simultaneously. In this one-step reaction, the same liquefact, enzymes and enzyme dosages were used as in Example 3. Temperature and pH were as in the first step of Example 3.

Samples were taken during the one-step reaction at appropriate times for HPLC and HPAE-PAD analysis as described, above. The sugar compositions of the final IMO syrups are shown in Table 9. Table 8 shows the composition of isomalto-oligosaccharides (% of total) as measured by HPAE-PAD and using the same calculations as described in Example 3. Panose is listed separately in Table 9 and subsequent Tables.

TABLE 8
Sugar profiles of IMO syrups after the one-step reaction

| | Enzyme | | | | | |
|---|---|---|---|---|---|---|
| Sugar | BBA | 2G | 626 | 4G | CspAmy | PF |
| DP1 | 38.4 | 35.6 | 27.3 | 24.3 | 25.2 | 22.5 |
| DP2 | 27.4 | 27.4 | 25.2 | 24.1 | 24.5 | 22.9 |
| DP3 | 15.2 | 16.4 | 18.8 | 19.4 | 18.6 | 18.5 |
| DP4 | 8.5 | 9.0 | 13.2 | 13.3 | 12.3 | 12.6 |
| DP5 | 3.7 | 4.3 | 8.7 | 9.2 | 7.9 | 8.6 |
| DP6 | 1.2 | 1.7 | 3.7 | 4.6 | 5.2 | 6.0 |
| DP7 | 0.4 | 0.7 | 1.6 | 2.1 | 3.0 | 4.0 |
| DP8 | 0.3 | 0.3 | 0.7 | 1.0 | 1.5 | 2.2 |
| DP9 | 0.2 | 0.2 | 0.3 | 0.5 | 0.7 | 1.1 |
| DP10 | 0.2 | 0.2 | 0.1 | 0.3 | 0.3 | 0.5 |
| DP11+ | 4.5 | 4.0 | 0.5 | 1.3 | 0.7 | 1.2 |

TABLE 9
IMO content of above syrups as measured by HPAE-PAD analysis.

| | Enzyme | | | | | |
|---|---|---|---|---|---|---|
| IMO | BBA | 2G | 626 | 4G | CspAmy | PF |
| IM2 | 16.6 | 16.3 | 14.5 | 13.9 | 14.5 | 13.4 |
| IM3 | 12.5 | 12.8 | 11.9 | 11.9 | 13.0 | 12.1 |
| Panose | 2.5 | 3.2 | 6.0 | 6.2 | 4.7 | 5.0 |
| IM4 | 7.8 | 7.8 | 7.7 | 7.8 | 8.6 | 8.6 |
| IM5 | 3.4 | 3.7 | 4.2 | 3.4 | 4.8 | 5.4 |
| IM6 | 1.2 | 1.7 | 3.5 | 4.4 | 4.6 | 5.2 |
| IM7 | 0.4 | 0.7 | 1.6 | 2.1 | 2.8 | 3.6 |
| Total IMO | 44.4 | 46.2 | 49.3 | 49.7 | 53.0 | 53.2 |

Comparing the final IMO syrups (at the end of the one-step reaction) that are summarized in Tables 8 and 9, it is apparent that pullulanse being active during TG reaction reduced the amount of DPn in all reactions, to levels much lower than in the two-step reaction. The degradation of higher sugars can clearly continue during the one-step reaction, resulting in better utilization of the available oligosaccharides.

As before, DP1 content decreases with increasing length of the donor molecule. Overall, more DP1 is formed in the one-step process compared to the two-step process. Even during transglycosylation with longer donor molecules, glucose is eventually released. DP2-DP5 content is similar in reactions with the DP3+ generating enzymes and a little lower than the reactions using the enzymes that generate maltose. DP6-DP10 content also increases with increasing length of the donor molecule in the one-step reaction, in the order: PF>CspAmy2>4G, 626>BBA/2G.

The amount of shorter IMO generally appears to decrease with increasing length of the donor molecule. The levels of isomaltotriose, isomaltotetraose and isomaltopentaose appear to remain approximately the same, while he levels of isomaltohexaose and isomaltoheptaose clearly increase with increasing length of the donor molecule.

Comparing the one-step reaction to the two-step reaction it is apparent that (a) more IMO is produced in the one-step reaction, (b) less panose is produced in the one-step reaction, (c) more isomaltose is produced in the one-step reaction and (d) more IM5, IM6 and IM7 are produced in the one-step reaction.

### Example 5. Desirable Amount of β-Amylase Activity

In the examples above, IMO production is performed without the use of a β-amylase and is compared to the traditional IMO production using of β-amylase. In this experiment it is investigated how much β-amylase activity can be present in the improved IMO production method without negating the benefits of the improved method. The one-step reaction with OPTMALT® 4G (without β-amylase), as described in Example 4, was repeated but with a dose-range of β-amylase present.

5 g of corn liquefact (DE 12.1, DS 33.1%) was incubated for 48 hr at 60° C. with the DP4-producing α-amylase (OPTIMALT® 4G) at 0.9 kg/MT DS with pullulanase (OPTIMAX® L2500) at 0.16 kg/MT DS and 1.0 kg/MT of transglucosidase at pH 5.0. The same reaction was performed with increasing amount of β-amylase (OPTIMALT® BBA) as indicated in the Table 10. As a control, the traditional one-step saccharification was performed with β-amylase (OPTIMALT® BBA) at 0.9 kg/MT, pullulanase (OPTIMAX® L2500) at 0.16 kg/MT and transglucosidase at 1.0 kg/MT at pH 5.0 and 60° C.

Samples were taken during the saccharification at appropriate times for HPLC and HPAE-PAD analysis as described in previous examples. The sugar compositions of the final IMO syrups are shown in Table 11. Table 12 shows the composition of isomalto-oligosaccharides (% of total) as measured by HPAE-PAD and using the same calculations as described in Example 3.

TABLE 10
Enzyme dosing for the determination of a desirable
amount of β-amylase

| | | | TG | |
|---|---|---|---|---|
| Reac- | OPTIMALT ® | OPTIMAX ® L- | L-2000 | OPTIMALT ® |
| tion | 4G kg/MT | 2500 kg/MT | kg/MT | BBA kg/MT |
| 1 | — | 0.16 | 1.0 | 0.90 |
| 2 | 0.9 | 0.16 | 1.0 | — |
| 3 | 0.9 | 0.16 | 1.0 | 0.01 |
| 4 | 0.9 | 0.16 | 1.0 | 0.02 |
| 5 | 0.9 | 0.16 | 1.0 | 0.05 |
| 6 | 0.9 | 0.16 | 1.0 | 0.10 |
| 7 | 0.9 | 0.16 | 1.0 | 0.20 |
| 8 | 0.9 | 0.16 | 1.0 | 0.50 |

TABLE 11
Sugar profiles (%) of reactions after 48 hours saccharification

| | Reaction | | | | | | | |
|---|---|---|---|---|---|---|---|---|
| Sugar | 1 | 2 | 3 | 4 | 5 | 6 | 7 | 8 |
| DP1 | 32.0 | 21.0 | 20.9 | 20.9 | 22.0 | 24.6 | 26.9 | 30.6 |
| DP2 | 26.4 | 21.7 | 21.9 | 22.5 | 23.3 | 24.6 | 25.8 | 27.6 |
| DP3 | 16.6 | 19.3 | 19.4 | 19.5 | 19.8 | 19.5 | 19.4 | 18.9 |
| DP4 | 9.8 | 14.0 | 14.0 | 14.1 | 13.9 | 13.2 | 12.7 | 11.7 |
| DP5 | 4.6 | 10.1 | 10.0 | 9.7 | 9.2 | 8.3 | 7.5 | 6.2 |
| DP6 | 1.6 | 5.4 | 5.3 | 5.0 | 4.6 | 4.0 | 3.3 | 2.4 |
| DP7 | 0.6 | 2.5 | 2.4 | 2.2 | 2.0 | 1.7 | 1.4 | 0.9 |
| DP8 | 0.4 | 1.2 | 1.2 | 1.1 | 0.9 | 0.8 | 0.6 | 0.4 |
| DP9 | 0.3 | 0.6 | 0.6 | 0.6 | 0.5 | 0.4 | 0.3 | 0.2 |
| DP10 | 0.3 | 0.7 | 0.7 | 0.6 | 0.5 | 0.6 | 0.5 | 0.2 |
| DP11+ | 7.4 | 3.6 | 3.6 | 3.7 | 3.2 | 2.2 | 1.6 | 0.8 |

TABLE 12
IMO content (%) of above syrups
as measured by HPAE-PAD analysis.

| | Reaction | | | | | | | |
|---|---|---|---|---|---|---|---|---|
| Sugar | 1 | 2 | 3 | 4 | 5 | 6 | 7 | 8 |
| IM2 | 19.8 | 14.5 | 15.8 | 16.6 | 17.2 | 18.2 | 19.3 | 21.1 |
| IM3 | 13.2 | 10.1 | 11.8 | 12.6 | 13.2 | 13.4 | 14.1 | 14.6 |
| Panose | 3.1 | 7.5 | 6.1 | 5.5 | 5.4 | 5.1 | 4.5 | 3.9 |
| IM4 | 9.6 | 12.5 | 12.5 | 12.4 | 12.5 | 12.2 | 11.9 | 11.3 |
| IM5 | 4.5 | 8.9 | 9.1 | 8.7 | 8.5 | 7.9 | 7.2 | 6.1 |
| IM6 | 1.6 | 5.4 | 5.3 | 5.0 | 4.5 | 4.0 | 3.3 | 2.4 |
| IM7 | 0.6 | 2.4 | 2.3 | 2.1 | 1.9 | 1.7 | 1.3 | 0.9 |
| Total IMO | 52.4 | 61.3 | 63.0 | 63.0 | 63.2 | 62.5 | 61.8 | 60.2 |

From Table 11 it is clear that, as seen in previous Examples, the reaction with OPTIMALT®4G produces a syrup having much lower DP1 than the traditional reaction with OPTIMALT® BBA. It also shows that a small amount of β-amylase can be present during the reaction with OPTIMALT® 4G without influencing the results significantly. Specifically, when there is up to 0.05 kg/MT OPTIMALT® BBA present, the DP1 is as low as when no BBA is present. Only when 0.1 kg/MT OPTIMALT® BBA is present, the DP1 begins to increase, and further increases, in a dose-dependent manner, according to the amount of the β-amylase. At a dose of 0.5 kg/MT the DP1 level is very close to that of the conventional reaction performed with only the β-amylase.

With the exception of DP11+, the amounts of other sugars produced follow the same trend. Up to a dose of 0.05 OPTIMALT® BBA, there is little change in the sugar profiles compared to the reaction without OPTIMALT® BBA. When the dose is further increased, the differences between the improved method and the conventional method become smaller with increasing dose. The amount of DP11+ is lower for the reactions where both OPTIMALT® 4G and OPTIMALT® BBA are used compared to the traditional reaction with only OPTIMALT® BBA. Apparently, the longer starch fragments are better hydrolysed when both enzymes are present.

As shown in Table 12, the amount of longer IMO (see, e.g., IM6 and IM7) is larger in the reaction with OPTIMALT® 4G than in the reaction with OPTIMALT® BBA. Also, in this example, less IM2 and more panose was measured. Overall, the total amount of IMO made in this experiment was higher than in previous experiments, which is likely due to the use of a different liquefact.

When a small amount of β-amylase (OPTIMALT® BBA) is present in the reaction along with OPTIMALT® 4G, IM2 is higher than without. The amount does not change with the dose up to a β-amylase dose of 0.05 kg/MT. At higher dosages of OPTIMALT® BBA the IM2 content increases to the same level as the conventional reaction with only OPTIMALT® BBA. Similar effects are seen for other sugars.

One surprising observation is that the total IMO content increases with 0.01 kg/MT of OPTIMALT® BBA present in the OPTIMALT® 4G reaction. As a small amount of β-amylase does not increase DP1 formation, it may actually be beneficial to the improved method. However, larger amounts of β-amylase are clearly incompatible with the present improved method.

### Example 6. Conversion of β-Amylase Dose to β-Amylase Activity Units

In the foregoing examples, enzyme dose is expressed in kg enzyme/MT substrate, which is convenient for commercial products, and is common practice in the industry. For the purpose of defining the improved method, the dose of β-amylase should be expressed in terms of activity units present in the reaction.

OPTIMALT® BBA has an average β-amylase activity of 1320 DP°/g product, where DP° refers to diastatic power. DP° determination is based on a 30-min hydrolysis of a starch substrate at pH 4.6 and 20° C. The reducing sugar groups produced upon hydrolysis are measured in a titrimetric procedure using alkaline ferricyanide. One unit of diastase activity, expressed as degrees DP (° DP), is defined as the amount of enzyme, contained in 0.1 ml of a 5% solution of the sample enzyme preparation, that will produce sufficient reducing sugars to reduce 5 mL of Fehling's solution when the sample is incubated with 100 mL of the substrate for 1 hour at 20° C. As an example, a dose of 0.05 kg/MT OPTIMALT® BBA is equivalent to 50 g/MT, which is equivalent to 50×1320 DP° units/MT, or 66,000 DP° units/MT. This amount is equivalent to 66 DP° units/kg, or 66 DP° units per kg of dry solids in a starch hydrolysate.

The results described in Example 5 indicate that the present improved method is not adversely affected by the presence of up to 66 DP° Units of β-amylase activity present per kg dry solids in the starch hydrolysate. With up to 66 DP° Units present, DP1 is as low as without β-amylase, with the same, or even higher, IMO content. It is estimated that the presence of as little as 13.2 DP°/kg may even be beneficial. However, when an increased amount of β-amylase activity is present, the benefits of the improved method is eroded, in a dose-dependent manner, until the IMO profile resemble that obtained by conventional method.

What is claimed is:

1. An improved method for producing isomalto-oligosaccharides (IMO) from maltodextrins, comprising the steps of:
(i) contacting maltodextrins with an α-amylase to produce malto-oligosaccharides, and
(ii) contacting the malto-oligosaccharides with a transglucosidase to produce IMO,
wherein the method produces longer chain IMO and/or reduced amounts of glucose compared to a method for producing IMO from maltodextrins using β-amylase in step (i).

2. The method of claim 1, wherein step (i) is performed in the presence of no more than 660 diastatic power (DP°) units β-amylase per kg dry weight maltodextrins.

3. The method of claim 1, wherein step (i) is performed in the presence of no more than 264 diastatic power (DP°) units per kg dry weight malto-oligosaccharides.

4. The method of claim 1, wherein step (i) is performed in the presence of no more than 132 diastatic power (DP°) units per kg dry weight malto-oligosaccharides.

5. The method of claim 1, wherein step (i) is performed in the presence of no more than 66 diastatic power (DP°) units per kg dry weight malto-oligosaccharides.

6. The method of claim 1, wherein step (i) is performed in the absence of a β-amylase.

7. The method of any of claims 1-6, wherein step (i) is performed using an α-amylase that produces malto-oligosaccharides that comprise at least 15% DP3.

8. The method of any of claims 1-7, wherein step (i) is performed using an α-amylase that produces malto-oligosaccharides that comprise at least 10% DP4.

9. The method of any of claims 1-8, wherein step (i) is performed using an α-amylase that produces malto-oligosaccharides that comprise at least 5% DP5.

10. The method of any of claims 1-9, wherein step (i) is performed using an α-amylase that produces malto-oligosaccharides that comprise no more than 40% DP2.

11. The method of any of claims 1-10, wherein step (i) is performed in the presence of a pullulanase.

12. The method of any of claims 1-11, wherein steps (i) and (ii) are performed sequentially.

13. The method of any of claims 1-11, wherein steps (i) and (ii) are performed simultaneously.

14. The method of any of claims 1-13, wherein the maltodextrins are prepared from a starch-containing substrate using a liquefying α-amylase.

15. The method of claim 14, wherein the liquefying α-amylase and the α-amylase used in step (i) are the same.

16. An improved method for producing isomalto-oligosaccharides (IMO), comprising the steps of (i) contacting a starch-containing substrate with a liquifying α-amylase to produce maltodextrins, (ii) contacting the maltodextrins with a DP3+ generating α-amylase to produce malto-oligosaccharides and (iii) contacting the malto-oligosaccharides with a transglucosidase to produce IMO having longer chains compared to IMO produced using β-amylase instead of DP3+ generating α-amylase in step (ii).

17. The method of claim 16, wherein the DP3+ generating α-amylase produces malto-oligosaccharides comprising at least 15% DP3, at least 10% DP4, at least 5% DP5, and/or no more than 40% DP2.

18. The method of claim 16 or 17, wherein steps (i) and (ii), and/or steps (ii) and (iii), are sequential, overlapping or simultaneous.

19. The method of any of claims 16-18, wherein step (ii) is performed in the presence of no more than 660, no more than 264, no more than 132, or no more than 66 diastatic power (DP°) units β-amylase per kg dry weight maltodextrins

20. The method of any of claims 16-19, wherein step (ii) is performed in the absence of a β-amylase.

21. The method of any of claims 16-20, wherein step (ii) is performed in the presence of a pullulanase.

22. The method of any of claims 16-21, wherein the liquefying α-amylase and the DP3+ generating α-amylase used in step (ii) are the same.

23. IMO produced by the method of any of claims 1-22.
